(19) United States
(12) Patent Application Publication
(10) Pub. No.: US 2017/0122541 A1
(43) Pub. Date: May 4, 2017

(54) Electronic Lighting Systems

(71) Applicant: Luminara Worldwide, LLC, Eden Prairie, MN (US)

(72) Inventors: Douglas Patton, Irvine, CA (US); Gary Bordenkicher, Lake Forest, CA (US); Channing Shattuck, Bonsall, CA (US); Jeffrey Thompson, Huntington Beach, CA (US)

(21) Appl. No.: 15/084,173

(22) Filed: Mar. 29, 2016

Related U.S. Application Data
(60) Provisional application No. 62/250,414, filed on Nov. 3, 2015.

(51) Int. Cl. F21V 23/06; F21S 6/00; A61L 9/12; F21S 9/02; F21V 33/00; F21V 23/02; F21S 10/04

(52) U.S. Cl. CPC F21V 23/06; F21V 23/02; F21S 6/001; F21S 10/046; F21S 9/02; F21V 33/0056; A61L 9/122; A61L 9/12; H04R 1/028

(57) ABSTRACT

An electronic lighting system comprising an electronic lighting device and a base device is disclosed. The electronic lighting device removably couples the base device to thereby provide power to the base device. More generally, an electronic system comprising a battery cover configured to couple a first electronic lighting device, and a second electronic device is disclosed. The battery cover comprises a battery contact and a first electrical contact, and the second electronic device comprises a second electrical contact. Power is provided to the second electronic device via the electrical coupling of the various electrical contacts.

This application claims priority to U.S. Provisional Application No. 62/250,414, filed Nov. 3, 2015. All extrinsic materials identified herein are incorporated by reference in their entirety.

### FIELD OF THE INVENTION

The field of the invention is electronic systems, and more particularly, electronic lighting systems having electronic lighting devices and a base device.

### BACKGROUND

The background description includes information that may be useful in understanding the present invention. It is not an admission that any of the information provided herein is prior art or relevant to the presently claimed invention, or that any publication specifically or implicitly referenced is prior art.

Electronic lighting systems have been used to provide light when needed. One example of a useful electronic lighting system is an electronic candle. Electronic candles are decorative and are much more convenient than a real candle. However, electronic candles are typically limited to providing light with no additional functionality.

To increase the functionality of the electronic candles, some have contemplated electronic candles that also emit fragrances. See, e.g., U.S. Pat. No. 7,350,720 to Jaworski et al.; US 2005/0285538 to Jaworski et al. (publ. December 2005); U.S. Pat. No. 7,481,571 to Bistritzky et al.; US 2008/0031784 to Bistritzky et al. (publ. February 2008); US 2006/0125420 to Boone et al. (publ. June 2006); US 2007/0127249 to Medley et al. (publ. June 2007); US 2008/0150453 to Medley et al. (publ. June 2008); US 2005/0169666 to Porchia, et al. (publ. August 2005); U.S. Pat. No. 7,503,668 to Porchia, et al.; U.S. Pat. No. 7,824,627 to Michaels, et al.; US 2006/0039835 to Nottingham et al. (publ. February 2006); US 2008/0038156 to Jaramillo (publ. February 2008); US 2008/0130266 to DeWitt et al. (publ. June 2008); US 2012/0024837 to Thompson (publ. February 2012); US 2011/0134628 to Pestl et al. (publ. June 2011); US 2011/0027124 to Albee et al. (publ February 2011); US 2012/0020052 to McCavit et al. (publ. January 2012); and US 2012/0093491 to Browder et al. (publ. April 2012).

Others have contemplated an electronic candle that is also rechargeable. For example, U.S. Pat. No. 9,039,227 to Fournier discloses a flameless candle having recharging contacts that may include electrically conductive material. A recharging port receives a bottom portion of the flameless candle having the recharging contacts to recharge the battery. Electrically coupling two devices has been discussed in other contexts, such as in U.S. Pat. No. 7,982,436 to Randall and US 2014/0132550 to McCracken.

These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

Although there have been advancements to increase the usefulness of electronic lighting system (and in particular electronic candles), the functionality of electronic lighting systems remains fairly limited. Thus, there is still a need for improved electronic lighting systems.

### SUMMARY OF THE INVENTION

The inventive subject matter provides apparatus, systems, and methods in which a base device may couple with an electronic lighting device to thereby provide electrical contact between the electronic lighting device and the base device. The electronic lighting device preferably comprises a first electrical contact and a power source, and the base device comprises a second electrical contact. It should be noted that the various electrical contacts disclosed herein comprise electrically conductive material. The electronic lighting device removably couples with the base device to provide electrical contact between the first electrical contact and the second electrical contact. Thus, the electronic lighting device is configured to provide power to the base device while the first electrical contact is in contact with the second electrical contact. Advantageously, this allows for additional functionality to be provided to the electronic lighting device. As just some examples, the base device could include a fragrance emitter, a wireless transmitter to receive information or instructions to operate the electronic lighting device, a speaker, a power source, and so forth.

It is contemplated that the first electrical contact comprises a projection, and the second electrical contact comprises an aperture or female end that is sized and dimensioned to receive the projection or a male end. The first electrical contact can be disposed on a base of the electronic lighting device. In some embodiments, the base device can comprise a fragrance emitter having a scented cartridge configured to produce scented air and a fan configured to move the scented air. The fragrance emitter can be powered by the power source via the first electrical contact and the second electrical contact.

It is further contemplated that the first electrical contact can comprise an arm and the second electrical contact can comprise a ring. Once again, the base device can comprise a fragrance emitter that is powered by the power source via the first electrical contact and the second electrical contact. The fragrance emitter comprises a scented cartridge configured to produce scented air, and a fan configured to move air within the fragrance emitter.

Although two electrical contacts have been described above, it is contemplated that the electronic lighting system can comprise additional electrical contacts. For example, the electronic lighting device can comprise a third electrical contact and the base device can comprise a fourth electrical contact. The electronic lighting device can be configured to couple the base device to provide contact between the third electrical contact and the fourth electrical contact to thereby provide power from the electronic lighting device to the base device.

When multiple electrical contacts are used, it is contemplated that power is provided to the base device when the first electrical contact is in contact with the second electrical contact, and the third electrical contact is in contact with the fourth electrical contact. However, it is also contemplated that power is provided to the base device when merely the first electrical contact is in contact with the second electrical contact, or the third electrical contact is in contact with the fourth electrical contact.

In another aspect, the inventors contemplate an electronic system comprising a battery cover configured to couple a first electronic device with a base unit (e.g., a second electronic device). The battery cover comprises a battery contact disposed on a first surface of the battery cover and a first electrical contact disposed on a second surface of the battery cover that can be opposite the first surface. The first electrical contact and the battery contact are electrically coupled. The second electronic device comprises a second electrical contact. The second electronic device is configured to receive power from the first electronic device when the first electrical contact contacts the second electrical contact and when the battery cover is coupled to the first electronic device.

In such embodiments, for example, an existing device (e.g., an electronic device having a conventional battery cover) can be retrofitted by swapping the original battery cover of the existing device with a new cover (e.g., battery cover described in the various embodiments) that allows for electrical coupling with a base device (e.g., second electronic device). In this manner, the base device can receive power from the existing device and thereby provide additional functionality to the existing device.

In contemplated embodiments, the first electrical contact comprises a projection, and the second electrical contact comprises an aperture that is sized and dimensioned to receive the projection. The first electronic device can comprise an electronic lighting device and the second electronic device can comprise at least one of a fragrance emitter, a speaker, and a transceiver. The electronic lighting device can comprise an outer shell, and a flame element disposed above the outer shell. In other contemplated embodiments, the first electrical contact comprises an aperture and the second electrical contact comprises a projection, wherein the aperture is configured to receive the projection.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a perspective view of an embodiment of an electronic lighting system comprising an electronic lighting device and a base device.

FIG. 2 is a cross-sectional view of a portion of the electronic lighting system of FIG. 1.

FIG. 3 is a bottom perspective view of the electronic lighting device of FIG. 1.

FIG. 4 is a top perspective view of the base device of FIG. 1.

FIG. 5 is a perspective view of another embodiment of an electronic lighting system comprising an electronic lighting device and a base device.

FIG. 6 is a cross-sectional view of a portion of the electronic lighting system of FIG. 5.

FIG. 7 is another cross-sectional view of a portion of the electronic lighting system of FIG. 5.

FIG. 8 is a bottom perspective view of the electronic lighting device of FIG. 5.

FIG. 9 is a perspective view of the base device of FIG. 5.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The inventor has discovered that electronic lighting systems can be improved to be more interactive and versatile. The electronic lighting system can include an electronic lighting device and a base device that can be electrically coupled to thereby transfer power from the electronic lighting device to the base device. In one arrangement, the electronic candle can be stacked on top of the base device to thereby provide power to the base device. It should be appreciated that the suitable base devices include at least one of a fragrance emitter, a speaker, and a transceiver to allow for wireless transmission or receipt of data including command signals. Thus, a retrofit or add-on is contemplated that allows for additional functionality of an electronic lighting device without the need to replace the electronic lighting device to obtain the additional functionality.

More generally, the inventor has discovered an electronic system comprising a battery cover that is configured to couple to a first electronic device that can be used to provide power to a second electronic device. The battery cover comprises electrical contacts that contact at least one electrical contact on the second electronic device to provide power to the second electronic device and increase the functionality of the first electronic device. In a contemplated embodiment, the first electronic device is an electronic lighting device and the second electronic device is a fragrance emitter. In such embodiment, the electronic lighting device provides power to the fragrance emitter to thereby provide a more versatile electronic lighting device that can emit fragrance. It should be appreciated that other second electronic devices can be used to provide other functions to the electronic lighting device (e.g., a speaker to play sound).

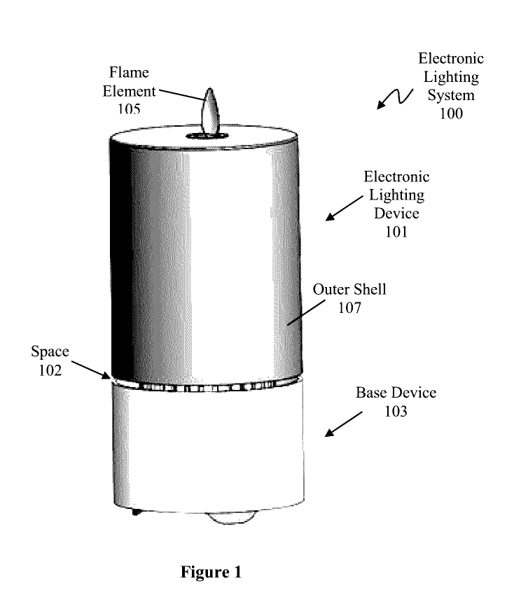

FIG. 1 shows an embodiment of an electronic lighting system 100 comprising a first electronic device as an electronic lighting device 101, and a second electronic device as a base device 103. Electronic lighting device 101 is a flameless candle comprising a flame element 105 and an outer shell 107. Flame element 105 extends through an aperture on outer shell 107, and flame element 105 is allowed to move in a manner that simulates a moving flame of a real candle. The mechanism that creates the motion of flame element 105 can vary. For example, a circuit board can control a drive mechanism, which could be an electromagnet, a fan, or other component that creates kinetic motion on flame element 105 to simulate the movement of a moving flame. A detailed description of an exemplary internal configuration for an electronic lighting device that is configured to move a flame element can be found in PCT International Application No. PCT/US2015/011642, which is hereby incorporated by reference.

Additionally, it is contemplated that flame element 105 can be supported within outer shell 107 using various suitable structures. Suitable structures to suspend flame element 105 include an arm that extends from a housing within the outer shell 107 or a support wire that extends through an aperture on flame element 105 to thereby suspend flame element 105. A detailed description of exemplary structures that can be used to suspend a flame element in an electronic lighting device can be found in U.S. application Ser. No. 14/819,146, which is hereby incorporated by reference.

Base device 103 can be a fragrance emitter having a space 102 to allow scented air to escape from base device 103 as shown in FIG. 1. In this way, a non-fragrance, electronic lighting device 101 can be converted into a fragrance candle. However, in other embodiments, it is contemplated that base device 103 comprises at least one of a fragrance emitter, speaker, an external battery and a transceiver. Thus, base device 103 can be interchanged with another base device to provide a desired function to a user. For example, if a user desires a scented candle, then the user can use a base device comprising a fragrance emitter, but if the user, or another user, later desires music, then the base device comprising the fragrance emitter can be interchanged with another base device comprising a speaker. It is also contemplated that base device 103 can comprise a plurality of a fragrance emitter, a speaker, an external battery and a transceiver, such that an individual base device can provide multiple functionalities (e.g., music and scented air, wireless communication and extended battery life, etc.).

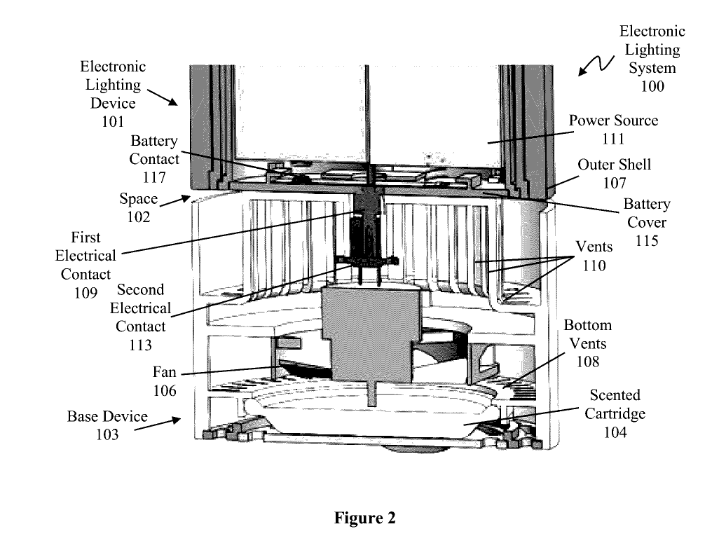

Electronic lighting device 101 comprises a first electrical contact 109 and a power source 111 as shown in FIG. 2. Power source 111 comprises at least one disposable battery (e.g., alkaline battery, lithium battery, etc.) that is housed within a battery holder of electronic lighting device 101. However, in other embodiments, it is contemplated that power source 111 can be a rechargeable battery.

Base device 103 comprises a second electrical contact 113. Electronic lighting device 101 is removably coupled with base device 103 to thereby provide electrical contact between first electrical contact 109 and second electrical contact 113 as shown in FIG. 2. Thus, electronic lighting device 101 can provide power to base device 103 when first electrical contact 109 contacts second electrical contact 113.

It is contemplated that power source 111 of electronic lighting device 101 provides all the power necessary for base device 103 to operate (i.e., base device 103 does not receive power from any other source). It should be appreciated that using electronic lighting device 101 to supply all the power necessary to operate base device 103 eliminates the need for a battery on base device 103. However, in other embodiments, electronic lighting device 101 can provide only part of the power necessary for base device 103 to operate or base device 103 can provide power to the electronic lighting device 101. Furthermore, in other embodiments, base device 103 can comprise a rechargeable battery that receives power from power source 111 to recharge the rechargeable battery, such that base device 103 can function when it is decoupled from electronic lighting device 101 using power from its rechargeable battery.

Electronic lighting device 101 can comprise a battery cover 115 that is removably coupled to electronic lighting device 101. Battery cover 115 comprises a battery contact 117 on an inside surface of battery cover 115. It is contemplated that battery contact 117 comprises an electrically conductive material. First electrical contact 109 can be at least partially disposed on an outside surface of battery cover 115. Thus, base device 103 can receive power from power source 111 when first electrical contact 109 contacts second electrical contact 113 and when the battery cover 115 is coupled to electronic lighting device 101 to thereby provide contact between battery contact 117 and power source 111. It is contemplated that battery cover 115 can be removed from electronic lighting device 101 to replace a battery cover of a third electronic device (e.g., a radio, flashlight, phone, another electronic lighting device and any other battery-operated device) and thereby allow the third electronic device to removably couple with base device 103 without the need to modify the third electronic device.

As discussed above, base device 103 can be a fragrance emitter that produces scented air. The fragrance emitter comprises a scented cartridge 104 configured to produce scented air and a fan 106 configured to move the scented air. Fan 106 can turn in either direction to cause air to move through base device 103 in either direction (e.g., up or down). Typically, scented air moves through bottom vents 108 and vents 110 before escaping through space 102 between electronic lighting device 101 and base device 103. It is contemplated that air can enter (or exit) via space 102. In embodiments where air enters via space 102, it exits through the bottom. In embodiments where fan 206 causes air to exit via the space 102, it enters through the bottom.

Fan 106 is powered by power source 111 of electronic lighting device 101. It should be noted that base device 103 does not have a battery to independently power fan 106, but instead, receives power from power source 111 to power fan 106. In other words, a circuit is completed to cause fan 106 within the base device 103 to turn when first electrical contact 109 electrically couples second electrical contact 113. However, in other embodiments, it is contemplated that base device 103 has its own power source and electronic lighting device 101 provides additional power to base device 103 or that base device 103 has its own power source and provides power to electronic lighting device 101.

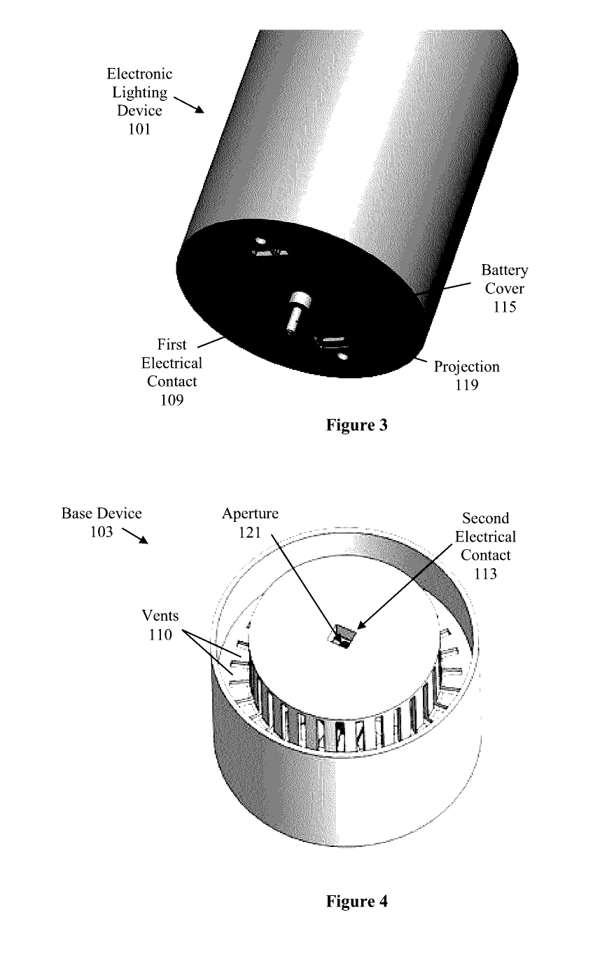

First electrical contact 109 comprises a projection 119 as shown in FIG. 3. Projection 119 extends away from an outside surface of battery cover 115. It is contemplated that first electrical contact 109 and projection 119 can be removed from electronic lighting device 101 by removing battery cover 115. Thus, battery cover 115 can be removed from electronic lighting device and can be coupled to third electronic device (e.g., a radio, flashlight, phone, another electronic lighting device and any other battery-operated device) to thereby provide a first electrical contact 109 and projection 119 to the third electronic device to removably couple with base device 103. As shown in FIG. 3, first electrical contact 109 is disposed on a base of electronic lighting device 101.

Electronic lighting device 101 can be coupled to base device 103, such that electronic lighting device 101 is stacked above base device 103. It is contemplated that second electrical contact 113 on base device 103 comprises an aperture 121 that is sized and dimensioned to receive projection 119 as shown in FIG. 4. Base device 103 is powered by power source 111 of electronic lighting device 101 when first electrical contact 109 contacts second electrical contact 113 via projection 119 being received by aperture 121.

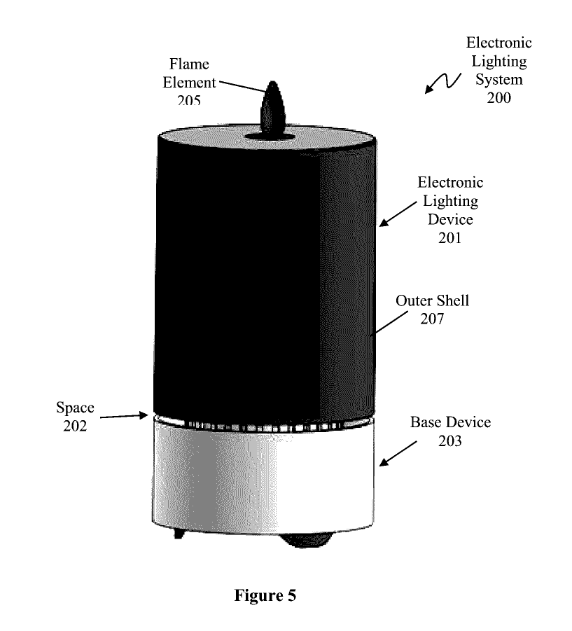

In another contemplated embodiment, an electronic lighting system 200 is disclosed comprising a first electronic device as an electronic lighting device 201, and a second electronic device as a base device 203 as shown in FIG. 5. Electronic lighting device 201 is a flameless candle comprising a flame element 205 and an outer shell 207. Flame element 205 extends through an aperture on outer shell 207, and is allowed to move freely in a manner that mimics a real candle light. The mechanism that creates the motion of flame element 205 and the structure that supports flame element 205 can vary as described above with respect to flame element 105.

Base device 203 can be a fragrance emitter having a space 202 to allow scented air to escape from base device 203 as shown in FIG. 5. However, in other embodiments, it is contemplated that base device 203 comprises at least one of a fragrance emitter, speaker, an external battery and a transceiver. Thus, base device 203 can be interchanged to provide various functionalities as desired by a user. For example, as discussed above, if a user desires a scented candle, then the user can use a base device comprising a fragrance emitter, but if the user, or another user, later desires music, then the base device comprising the fragrance emitter can be interchanged with another base device comprising a speaker.

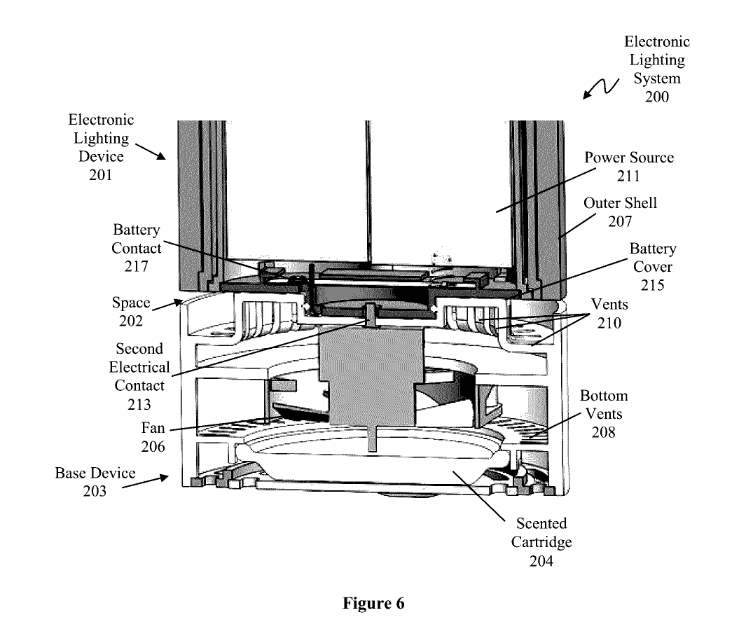

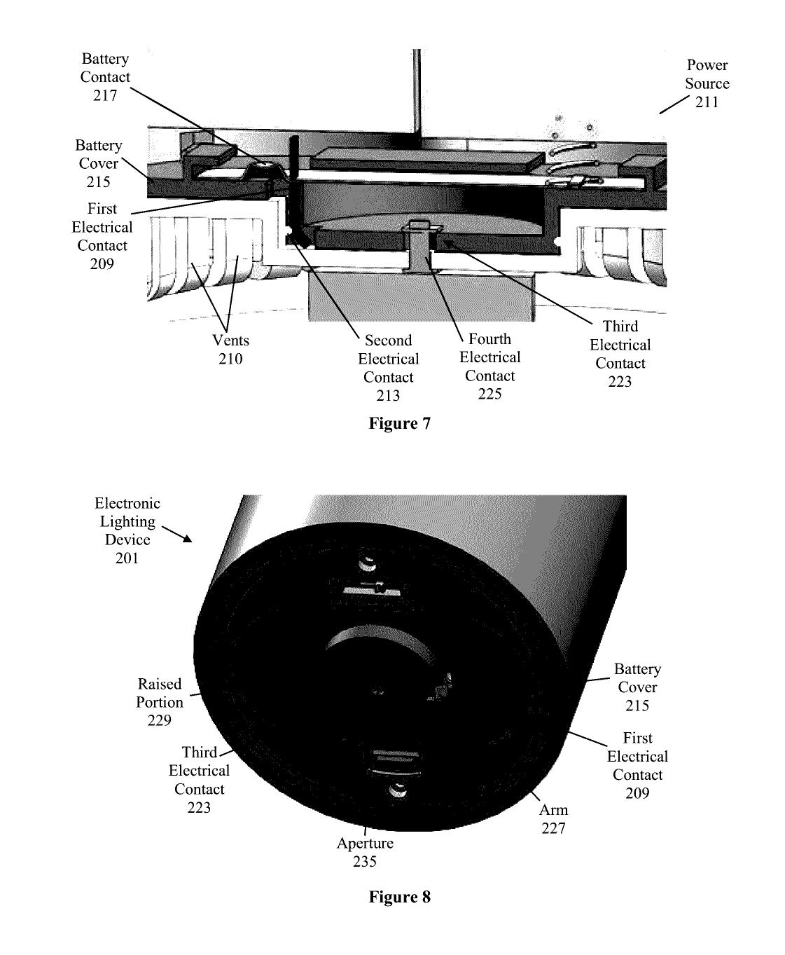

Electronic lighting device 201 comprises a first electrical contact 209 and a power source 211 as shown in FIGS. 6 and 7. Power source 211 comprises at least one disposable battery (e.g., alkaline battery, lithium battery, etc.) that is housed within a battery holder of electronic lighting device 201. However, in other embodiments, it is contemplated that power source 111 can be a rechargeable battery.

Base device 203 comprises a second electrical contact 213. As disclosed in the embodiment above, electronic lighting device 201 and base device 203 are removably coupled. In its coupled configuration, first electrical contact 209 electrically contacts second electrical contact 213 to thereby provide power from power source 211 of electronic lighting device 201 to base device 203. In its decoupled configuration, first electrical contact 209 does not contact second electrical contact 213, and no power is provided from power source 211 of electronic lighting device 201 to base device 203 via first electrical contact 209 and second electrical contact 213.

Furthermore, it is contemplated that electronic lighting device 201 further comprises a third electrical contact 223, and base device 203 further comprises a fourth electrical contact 225. Similar to first electrical contact 209 and second electrical contact 213, power from power source 211 of electronic lighting device 201 is provided to base device 203 when third electrical contact 223 contacts fourth electrical contact 225. It is contemplated that contact between (i) first electrical contact 209 and second electrical contact 213, and (ii) third electrical contact 223 and fourth electrical contact 225 is needed to provide power from power source 211 of electronic lighting device 201 or that merely contact between (i) first electrical contact 209 and second electrical contact 213, or (ii) third electrical contact 223 and fourth electrical contact 225 is sufficient to provide power from power source 211 of electronic lighting device 201 to base device 203.

Similar to electronic lighting system 100, it is contemplated that power source 211 of electronic lighting device 201 provides all the power necessary to operate base device 203 (i.e., base device 203 does not receive power from any other source). It should be appreciated that using electronic lighting device 201 to supply all the power necessary to operate base device 203 eliminates the need for a battery on base device 203. However, in other embodiments, electronic lighting device 201 can provide only part of the power necessary for base device 203 to operate or base device 203 can provide power to the electronic lighting device 201. Also, as discussed above, it is contemplated that base device 203 can comprise a rechargeable battery that receives power from power source 211 to recharge the rechargeable battery, such that base device 203 can function when it is decoupled from electronic lighting device 201 using power from its rechargeable battery.

Base device 203 can be a fragrance emitter. With respect to the features of the fragrance emitter, the characteristics described with respect to scented cartridge 104, fan 106, bottom vents 108, vents 110 are applicable to a scented cartridge 204, a fan 206, bottom vents 208, and vents 210, respectively.

Fan 206 can be solely powered by power source 211 of electronic lighting device 201 by receiving power from power source 211. In other words, a circuit is completed to cause fan 206 within the base device 203 to turn when first electrical contact 209 electrically couples second electrical contact 213, and third electrical contact 223 electrically couples fourth electrical contact 225. However, it is also contemplated that electrical coupling of first electrical contact 209 and second electrical contact 213 or third electrical contact 223 and fourth electrical contact 225 is sufficient to complete the circuit and cause fan to turn. Furthermore, in other contemplated embodiments, fan 206 can be partially powered by a power source of base device 203 and receive supplemental power from power source 211 of electronic lighting device 201.

A battery cover 215 is removably coupled to electronic lighting device 201 that maintains power source 211 within the body of electronic lighting device 201. Battery cover 215 comprises a battery contact 217 on an inside surface of battery cover 215 that is electrically coupled with power source 211. It is contemplated that battery contact 217 has electrically conductive material and that battery contact 217 is electrically coupled to at least one of first electrical contact 209, second electrical contact 213, third electrical contact 223, and fourth electrical contact 225. It is contemplated that battery cover 215 can be removed from electronic lighting device 201 to replace a battery cover of a third electronic device (e.g., a radio, flashlight, phone, another electronic lighting device and any other battery-operated device) and thereby allow the third electronic device to removably couple with base device 203 without the need to modify the third electronic device.

First electrical contact 209 and third electrical contact 223 are disposed on a base of electronic lighting device 201 as shown in FIG. 8. First electrical contact 209 comprises an arm 227 that extends away from the base of electronic lighting device 201 at an outward angle (i.e., extends partially towards outer shell 207). It is contemplated that arm 227 comprises electrically conductive material. Typically, first electrical contact 209 and arm 227 are electrically coupled with battery contact 217.

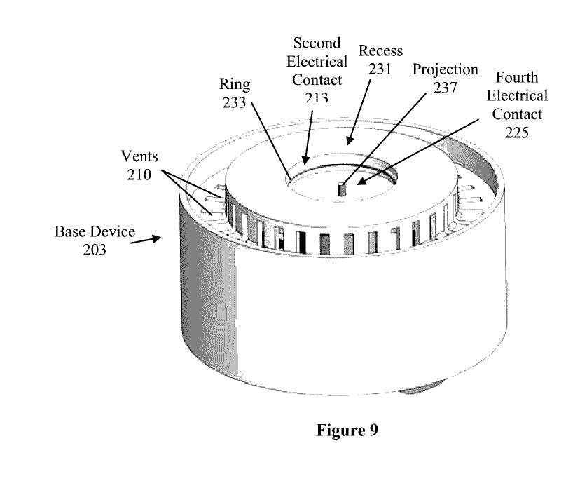

Battery cover 215 comprises a raised portion 229 having third electrical contact 223. Preferably, raised portion 229 is flat on the bottom to allow electronic lighting device 201 to sit flat on a surface when not coupled to base device 203. Raised portion 229 of battery cover 215 is received by a recess 231 (shown in FIG. 9) of base device 203. Recess 231 comprises second electrical contact 213 and fourth electrical contact 225. When raised portion 229 is received by recess 231, electrical contact is established between first electrical contact 209 and second electrical contact 213, and third electrical contact 223 and fourth electrical contact 225 to thereby provide power from power source 211 to base device 203.

As discussed above, first electrical contact 209 can comprise an arm 227. It is contemplated that in such embodiments, second electrical contact 213 can comprise a ring 233. Arm 227 can extend outwardly (i.e., at least partially towards outer shell 207) beyond the outer circumference of raised portion 229, such that arm 227 acts as a detent when it is received within recess 231. It is also contemplated that arm 227 can be received by a female portion (e.g., a keyhole, a notch, etc.) to lock base device 203 with electronic lighting device 201. For example, base device 203 can have a keyhole configured to receive arm 227, and electronic lighting device 201 can be oriented to align the keyhole with arm 227 to couple the keyhole with arm 227 and lock electronic lighting device 201 with base device 203. In another example, base device can have a keyhole configured to receive arm 227, and electronic lighting device 201 can be stacked onto base device 203 in any orientation and, when stacked, electronic lighting device 201 can be twisted/turned to couple arm 227 with the keyhole to lock electronic lighting device 201 and base device 203.

Ring 233 is circumferentially disposed within recess 231. When raised portion 229 is received by recess 231, arm 227 contacts ring 233 to provide electrical contact and thereby provide power from power source 211 to base device 203. The resistance to coupling provided by arm 227 ensures a good electrical connection between ring 233 and arm 227 by causing the electronic lighting device 201 to snap into base device 203 such that arm 227 is constantly pressing against ring 233. In some embodiments, once the ring 233 is electrically coupled to arm 227, a circuit is closed that allows electricity to flow from power source 211 of electronic lighting device 201 to fan 206 within base device 203.

Furthermore, third electrical contact 223 can comprise an aperture 235, and fourth electrical contact 225 can comprise a projection 237. It is contemplated that aperture 235 is configured to receive projection 237 to provide electrical contact between third electrical contact 223 and fourth electrical contact 225 to thereby provide power from power source 211 to base device 203.

As discussed above, electronic lighting device 201 comprises first electrical contact 209 and third electrical contact 223, and base device comprises second electrical contact 213 and fourth electrical contact 225. However, in other contemplated embodiments, electronic lighting device 201 can comprise only one of first electrical contact 209 or third electrical contact 223, and base device 203 can comprise only one of second electrical contact 213 or fourth electrical contact 225.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Also, as used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. An electronic lighting system, comprising:
an electronic lighting device comprising a first electrical contact and a power source;
a base device comprising a second electrical contact;
wherein the electronic lighting device is configured to removably couple with the base device to provide electrical contact between the first electrical contact and the second electrical contact; and
wherein the electronic lighting device is configured to provide power to the base device when the first electrical contact contacts the second electrical contact.

2. The system of claim 1, wherein the first electrical contact comprises a projection, and the second electrical contact comprises an aperture that is sized and dimensioned to receive the projection.

3. The system of claim 2, wherein the first electrical contact is disposed on a base of the electronic lighting device.

4. The system of claim 2, wherein the base device comprises a fragrance emitter.

5. The system of claim 4, wherein the fragrance emitter is powered by the power source via the first electrical contact and the second electrical contact.

6. The system of claim 4, wherein the fragrance emitter comprises a scented cartridge configured to produce scented air, and a fan configured to move the scented air.

7. The system of claim 1, wherein the first electrical contact comprises an arm and the second electrical contact comprises a ring.

8. The system of claim 7, wherein the electronic lighting device further comprises a third electrical contact and the base device further comprises a fourth electrical contact, and wherein the electronic lighting device is configured to couple the base device to provide contact between the third electrical contact and the fourth electrical contact to thereby provide power from the electronic lighting device to the base device when the third electrical contact contacts the fourth electrical contact.

9. The system of claim 8, wherein the first electrical contact and the third electrical contact are disposed on a base of the electronic lighting device, and wherein the fourth electrical contact comprises a projection and the third electrical contact comprises an aperture that is sized and dimensioned to receive the projection.

10. The system of claim 7, wherein the base device comprises a fragrance emitter.

11. The system of claim 10, wherein the fragrance emitter is powered by the power source via the first electrical contact and the second electrical contact.

12. The system of claim 11, wherein the fragrance emitter comprises a scented cartridge configured to produce scented air, and a fan configured to move air within the fragrance emitter.

13. The system of claim 7, wherein the electronic lighting device comprises raised portion and the base device comprises a recess that is sized and dimensioned to receive the raised portion.

14. The system of claim 13, wherein the ring is disposed in the recess, and wherein the arm extends outwardly to thereby provide a press fit between the arm and the ring when the recess receives the raised portion.

15. An electronic system, comprising:
a battery cover configured to couple a first electronic device, wherein the battery cover comprises a battery contact disposed on a first surface of the battery cover;
a first electrical contact disposed on a second surface of the battery cover, wherein the first electrical contact is electrically coupled to the battery contact;
a second electronic device comprising a second electrical contact, wherein the second electronic device is configured to receive power from the first electronic device when the first electrical contact contacts the second electrical contact and when the battery cover is coupled to the first electronic device.

16. The system of claim 15, wherein the first electrical contact comprises a projection, and the second electrical contact comprises an aperture that is sized and dimensioned to receive the projection.

17. The system of claim 16, wherein the first electronic device comprises an electronic lighting device and the second electronic device comprises at least one of a fragrance emitter, a speaker, and a transceiver.

18. The system of claim 15, wherein the first electronic device is an electronic lighting device, and wherein the battery cover removably couples to the electronic lighting device.

19. The system of claim 18, wherein the electronic lighting device comprises an outer shell, and a flame element disposed above the outer shell.

20. The system of claim 15, wherein the first electrical contact comprises an aperture and the second electrical contact comprises a projection, wherein the aperture is configured to receive the projection.
